(19) United States
(12) Patent Application Publication
(10) Pub. No.: US 2012/0289885 A1
(43) Pub. Date: Nov. 15, 2012

(54) PHOTOTHERAPY SYSTEM

(76) Inventors: William Jude Cottrell, Somerville, MA (US); Nathan Guild Kennedy, North Andover, MA (US)

(21) Appl. No.: 13/469,977

(22) Filed: May 11, 2012

Related U.S. Application Data
(60) Provisional application No. 61/518,907, filed on May 14, 2011.

(51) Int. Cl. A61M 37/00; A61N 5/06

(52) U.S. Cl. 604/20; 607/88

(57) ABSTRACT

The present application is directed to a phototherapy system for treating a treatment region having a treatment surface. The phototherapy system comprises an optical device. The optical device includes a light source generating source light, an emission surface emitting emitted light, and a sheet waveguide. The light source is coupled to direct the source light into the sheet waveguide. The sheet waveguide has a plurality of light extraction features that direct light out of the sheet waveguide. The emitted light has a wavelength to activate a photoactive compound; the emitted light has a therapeutic radiant emittance. The phototherapy system may comprise a feedback component that measures the light intensity near the treatment surface and provide feedback to adjust the source light. The phototherapy system may further comprise a treatment patch and a photoactive compound within the treatment region.

### RELATED APPLICATIONS

This application claims the benefit of priority of U.S. Provisional Patent Application No. 61/518,907, filed May 14, 2011 entitled “Phototherapy System”, which is incorporated herein by reference.

### FIELD

This patent application generally relates to a phototherapy system for delivering a therapeutic affect to a treatment region. More specifically, it relates to a phototherapy system that comprises a light emitting surface coupled with at least one from the group including a treatment patch and a therapeutic compound.

### BACKGROUND

The targeted delivery of light to tissue has been used for centuries to deliver both therapeutic and cosmetic results. This type of therapy using light is often referred to generally as phototherapy. There are a wide variety of phototherapy treatments which do not require exogenous compounds, but rather rely primarily on just the delivery of light to the treatment region. These therapies include photorejuvination, laser hair removal, tattoo removal, treatments of neonatal jaundice, psoriasis, and acne, just to name a few. Additionally, there is a class of phototherapy that includes the application of a drug or other agent. Photodynamic therapy (PDT) is among this class and uses the interaction of light, a photosensitizer and oxygen to cause a localized therapeutic effect. PDT has been shown to be effective in treating many indications, most notably cancer.

Light dose rate and total light dose delivered to a target region are important parameters for many phototherapies. Light dose rate is often specified as the radiant energy fluence rate (Watts/meter²). Total light dose can be specified as the total fluence (Joules/meter²). Interchangeably with fluenceate, the term irradiance is used for characterizing dose rate. A specific combination of fluence rate and fluence specifies the light delivery protocol for a phototherapy.

In the case of PDT, accurate and predictable delivery of light to a target tissue has been shown to dramatically impact treatment efficacy, tolerability, and efficiency. The impact of the tissue optical properties on the distribution of light in a target region has been investigated for decades (Patterson and Chance). The primary properties that govern the propagation of light in tissue are absorption, scattering, tissue geometry and the index of refraction of the tissue. These properties can vary significantly throughout a tissue. Tissue optical properties can often be approximated as a homogenous material with a characteristic absorption and scattering coefficients μₐ and μₛ, respectively, and refractive index n (Finlay).

Determining fluence rates in-vivo can be a complicated problem in many instances due to complex tissue geometries and heterogeneities. In the case of PDT treatments for Barrett's Esophagus (BE), for example, a diffusing fiber was centered inside a throat using a balloon. Treatment fluence rate was initially calculated by dividing the total output power of the diffuser by the treatment area (Gossner et al.). A more accurate calculation and measurement of delivered fluence rate, however, revealed that back scattering and reflection in the esophagus dramatically increased the light dose deposited by up to 3.9-fold (Bays et al. and van Veen et al.). The same effects of back scattering and reflective contributions have also been noted during PDT treatments involving other hollow organs including the bladder (van Staveren et al.) and the bronchi (Murrer et al.). Reflection from tissue surface and other tissue properties also affect delivery of PDT treatment, prompting some to examine in-vivo light dose directly (Marijnissen and Star). The impact of backscatter and optical resonance, however, is largely ignored in many treatments, particularly in dermatological applications which have traditionally relied on stand-off light sources and where the source and tissue geometries are often less complicated.

Recent advances in source technology, however, have brought light sources into contact and near-contact geometries, which have advantages in both convenience and size. In U.S. Pat. No. 7,686,839, Parker discloses a single source light emitter that comprises a plurality of optical fibers which effectively illuminates a large treatment area. In addition, conformal light diffusers which conform to the contours of a treatment area have also been described by Zhu et al. Zhu uses a laser source diffused through an intralipid liquid volume to provide a somewhat uniform and conformal illumination source.

Additional compact light sources using large area diffusers, originally developed for liquid crystal display backlighting purposes, have been available since the 1990's. Tagaya et al. describes “a light-guide plate LGP, which is typically a wedge-shaped polymer plate, has been widely used in the backlights for LCDs of portable devices the LGP is a device that converts a linear light source or a point light source into an area light source.” In US Patent Application Publication 2009/0198173, Samuel et al. modify an LGP, referred to therein as a “diffusing member”, making the diffusing member circular to illuminate an area of tissue. Backlighting innovations have led to the production of even area illumination using thin waveguides with patterned reflective and scattering features (Cassarly et al.). Backlighting innovations have also improved directionality using additional polymer layers, such as brightness enhancing films, to direct the photons toward the eye, U.S. Pat. No. 6,760,157 to Allen et al.

In surface delivery therapies, such as dermatological applications of PDT, the light dose and light dose rate over a given target region is extrapolated from light source parameters or measured in a free space arrangement. In light delivery geometries, however, the contribution of backscattered and reflected light from the target region itself can influence the fluence rate and impact the total phototherapy fluence over the treatment region.

In phototherapy, the selected wavelengths of light are often very important for obtaining a therapeutic affect. Tissue optical properties such as absorption, scattering and refractive index can vary with wavelength and therefore the propagation, distribution and uniformity of light within a target region may vary. The wavelengths of light may also impact any phototherapy compounds which are used. In the case of photodynamic therapy where a photosensitizer is used, the absorption of light by the photosensitizer depends on the wavelength of light used to activate the photosensitizer. Therefore, proving a phototherapy treatment using the same fluence rate with different wavelengths may provide different therapeutic effects. In the art, local maximums in a specific photosensitizer's absorption spectrum are called activation wavelengths. As an illustrative example, for aminolevulinic acid PDT therapy where the photosensizer is protoporphyrin IX, activation wavelengths occur near 405 nm and 633 nm.

Prior art shows that many parameters play a role in achieving the best phototherapeutic affect to a treatment region. The current application puts forth a novel structure and method that takes into account these parameters and provides a phototherapy system that provides more effective therapy to a treatment region.

### SUMMARY

One aspect of the present patent application is directed to a phototherapy system for a treatment region having a treatment surface. The phototherapy system comprises an optical device. The optical device includes a light source generating source light and a sheet waveguide. The sheet waveguide has a pair of sheet surfaces. The light source is coupled to direct the source light into the sheet waveguide. A plurality of light extraction features exist on at least one of each pair of sheet surfaces. The optical device further includes an emission surface emitting emitted light. The emitted light has a wavelength to activate a photoactive compound. The emitted light has a therapeutic radiant emittance. The phototherapy system may further comprise a feedback component that measures the light intensity near the treatment surface and provide feedback to adjust the source light. The phototherapy system may further comprise a treatment patch having a photoactive compound within the treatment region.

Another aspect of the present patent application is directed to a phototherapy system for a treatment region having a treatment surface. The phototherapy system comprises an optical device having a light source generating source light and an emission surface emitting emitted light. The emission surface has an initial radiant emittance. When the emission surface is coupled with the treatment region, the emitted light in combination with reflected light and scattered light from the treatment region sum to create an irradiance on the treatment surface that is greater than 1.5 times the initial radiant emittance.

Another aspect of the present patent application is directed to a phototherapy system for a treatment region having a treatment surface. The phototherapy system comprises an optical device having a light source generating source light and an emission surface emitting emitted light. The emitted light has a heterogeneous intensity distribution. When the emission surface is couple with the treatment region, the emitted light in combination with reflected light and scattered light from the treatment region sum to create a total light intensity distribution that is homogeneous over the treatment surface of the treatment region.

Another aspect of the present patent application is directed to a phototherapy system for a treatment region having a treatment surface. The phototherapy system comprises an illuminating layer having a light source and an emission surface emitting emitted light. The phototherapy system further comprises a treatment patch having a top patch surface and an aperture therein. The illuminating layer is coupled to the top patch surface of the treatment patch such that emitted light from the emission surface enters the aperture. The emitted light has a wavelength to activate a photoactive compound. The emitted light has a therapeutic radiant emittance.

Still another aspect of the present patent application is directed to a method of delivering phototherapy to a treatment region having a treatment surface. The method comprises providing: i) a light source generating source light with a source radiant emittance, ii) a detector, and iii) a feedback loop between the detector and the light source. The method further comprises coupling the light source with the treatment region so that the source light in combination with reflected light and scattered light from the treatment region sum to create a combined radiant emittance greater than 1.5 times the source radiant emttance and then generating a desired fluence rate from the light source light upon the treatment surface. The method further comprise measuring fluence rate provided to the treatment region by the detector and then delivering a desired light delivery protocol to the treatment surface based on feedback from the feedback loop.

Yet another aspect of the present patent application is directed to a method of delivering phototherapy to a treatment region having a treatment surface. The method comprises providing: i) optical parameters of the treatment region, ii) a light source generating source light with a set of optical parameters, and iii) a desired light distribution over the treatment region. The method further comprises modeling a distribution of light extraction features based on source light from the light source, reflected light from the treatment region and scattered light from the treatment region which, in combination with the light source, produce the desired light distribution. The,method further comprises building an optical device using the distribution of light extraction features and then delivering phototherapy to the treatment region using the optical device.

Still yet another aspect of the present patent application is directed to a method of fabricating a phototherapy system for a treatment region. The method comprises providing: i) optical parameters of the treatment region, ii) a light source generating source light with a set of optical parameters, and iii) a desired total light irradiance distribution over the treatment region. The method further comprises calculating a distribution of light extraction features based on source light from the light source, reflected light from the treatment region and scattered light from the treatment region; and then fabricating a device having the distribution of light extraction features to give the desired light intensity distribution over the treatment region when the optical device is couple with the treatment region.

### BRIEF DESCRIPTION OF DRAWINGS

The foregoing and other aspects and advantages will be apparent from the following detailed description, as illustrated in the accompanying drawings, in which:

FIG. 1a is a perspective view of one embodiment of the phototherapy system;

FIG. 1b is a partial cut away, perspective view of the phototherapy system in FIG. 1a showing the inner elements of the optical device, the treatment patch and the treatment region;

FIG. 2 is an exploded, partial cut away, perspective view of the phototherapy system in FIG. 1a;

FIG. 3a is a perspective view of the optical device in FIG. 1a;

FIG. 3b is a perspective view of the optical device in FIG. 3a with the outer casing removed;

FIG. 4 is an inverted, exploded perspective view of the components of the optical device in FIG. 3b;

FIG. 5 is an inverted, exploded perspective view of the diffuser assembly of the optical device in FIG. 3b;

FIG. 6a is a side cross-section view of components of the optical device in FIGS. 3a and 3b showing how source light interacts with the diffuser assembly components to generate emitted light;

FIG. 6b is a side cross-section view of components of the optical device in FIGS. 3a and 3b showing the optical device near the treatment region and how emitted light interacts with a treatment region;

FIG. 7a is a bottom view of a waveguide for an embodiment of the optical device in FIGS. 3a and 3b, showing a potential distribution of extraction features;

FIG. 7b is a bottom view of a waveguide for an embodiment of the optical device in FIGS. 3a and 3b, showing a potential distribution of extraction features;

FIG. 8a is a bottom view of an emission map for an embodiment of the optical device shown in FIGS. 3a and 3b;

FIG. 8b is a representative emission line scan plot for the emission map in FIG. 8a;

FIG. 8c is a bottom view of a partially transmissive filter optical density map for an embodiment of the optical device shown in FIGS. 3a and 3b;

FIG. 8d is a representative optical density line scan plot for the emission map in FIG. 8c;

FIG. 8e is a bottom view of an emission map for an embodiment of the optical device shown in FIGS. 3a and 3b;

FIG. 8f is a representative emission line scan plot for the emission map in FIG. 8e;

FIG. 9a is a bottom view of an emission map for an embodiment of the optical device shown in FIGS. 3a and 3b;

FIG. 9b is a representative emission line scan plot for the emission map in FIG. 9a;

FIG. 9c is a bottom view of an emission map for an embodiment of the optical device shown in FIGS. 3a and 3b;

FIG. 9d is a representative emission line scan plot for the emission map in FIG. 9c;

FIG. 10a is an exploded, perspective view of the phototherapy system in FIG. 1a showing alignment of a treatment aperture with the treatment region;

FIG. 10b is side cross-section view of the optical device shown in FIGS. 3a and 3b aligned to the treatment aperture of the phototherapy system in FIG. 1a;

FIG. 11a is a perspective view of an embodiment of the treatment patch used in FIG. 1a showing a protective layer;

FIG. 11b is a perspective view of an embodiment of the optical device shown in FIGS. 3a and 3b used in FIG. 1a showing a protective layer;

FIG. 12 is a perspective view of an embodiment of the phototherapy system where the edges of the diffuser assembly extend beyond the edges of the source assembly of the optical device;

FIG. 13a is a perspective view of an embodiment of the phototherapy system in FIG. 1a where the treatment patch includes cooperative depressions that engage with complementary ridges on the optical device;

FIG. 13b is a side, cross-section view of the phototherapy system in FIG. 13a where the treatment patch includes cooperative depressions that engage with complementary ridges on the optical device;

FIG. 14a is a side, cross-section view of an embodiment of the phototherapy system of FIGS. 1a and 1b with the light source now replaced by a remote source light tethered to the optical device through an optical fiber;

FIG. 14b is a partial cut away, perspective view of the phototherapy system in FIG. 14a showing the inner elements of the optical device, the treatment patch and the treatment region;

FIG. 15a is a side, cross-section view of an embodiment of the phototherapy system where the diffuser assembly of FIG. 1a and 1b is constructed from thin films and the rest of the optical components are now replaced by a remote source light tethered to the optical device through an optical fiber;

FIG. 15b is a partial cut away, perspective view of the phototherapy system in FIG. 15a showing the inner elements of the optical device, the treatment patch and the treatment region;

FIG. 16a is an inverted, exploded, perspective view of an alternative embodiment of the phototherapy system of FIGS. 14a and 14b;

FIG. 16b is a bottom view of a waveguide for an embodiment of the optical device in FIG. 16a, showing a potential distribution of extraction features and a lenticular source coupler;

FIG. 17a is a perspective view of an embodiment of the phototherapy system where multiple optical devices of those shown in FIGS. 3a and 3b are joined together to provide emitted light to a treatment region; and

FIG. 17b is a side, cross-section view of the phototherapy system in FIG. 17a.

### DETAILED DESCRIPTION

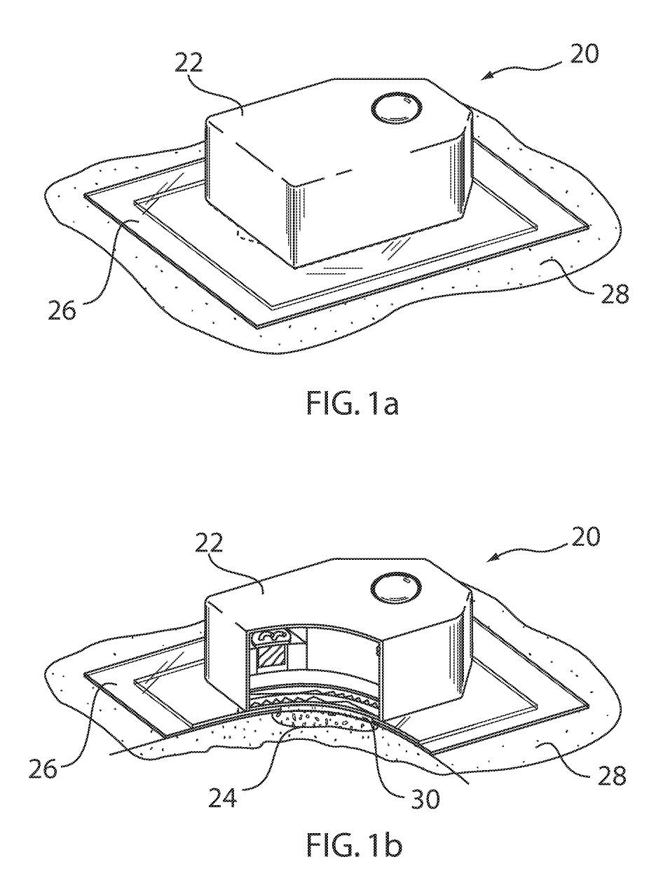

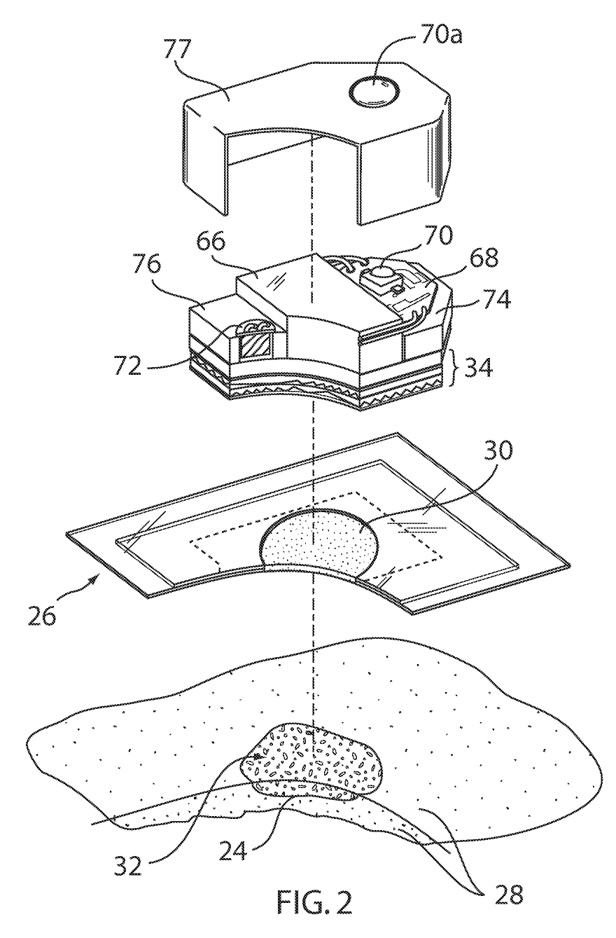

The present invention is illustrated in FIGS. 1a-17b. Phototherapy system 20, FIGS. 1a, 1b and 2, comprises an optical device 22 for providing a therapeutic affect to a treatment region 24. Treatment region 24 resides within a non-treatment region 28. Treatment region 24 has a treatment surface 32. Treatment region 24 is at least one from the group including cells, tissues and organs. Phototherapy system 20 may further comprise a treatment patch 26. Treatment patch 26 is used in combination with optical device 22 to maintain optical device 22 in near contact with treatment region 24 and provide other benefits such as helping isolate the therapeutic affect to the treatment region and providing alignment of optical device 22 to the treatment region during application. Phototherapy system 20 may still further comprise a treatment compound 30 supplied to the treatment region 24, which enhances the treatment affect. Treatment compounds 30 may be at least one from the group including an anesthetic, photoactive compound, index matching material, antibiotics, anti-inflammatory drugs, thermal compound and other active or inactive compounds. Further, photoactive compounds may be photosensitizers, sensitizer pro-drugs, fluorophores, chromophores, semiconductors, nanoparticles, and plasmon nanoparticles. In the case of photodynamic therapy (PDT), for example, the treatment compound may comprise a photosensitizer in a carrier cream.

Having a well controlled intensity distribution of light incident upon treatment surface 32 of treatment region 24 is important for providing an effective phototherapeutic treatment. This is accomplished by providing an optical device 22 that when in near proximity with the treatment region the optical device takes into account many parameters that affect light delivery. These parameters include the source light distribution and intensity, reflected light from the treatment region, scattered light from the treatment region, mitigation of stray light outside the treatment region, occlusion of exogenous light and controlling the temperature of the treatment region. The applicants have found certain desirable configurations for optical device 22 that address these issues.

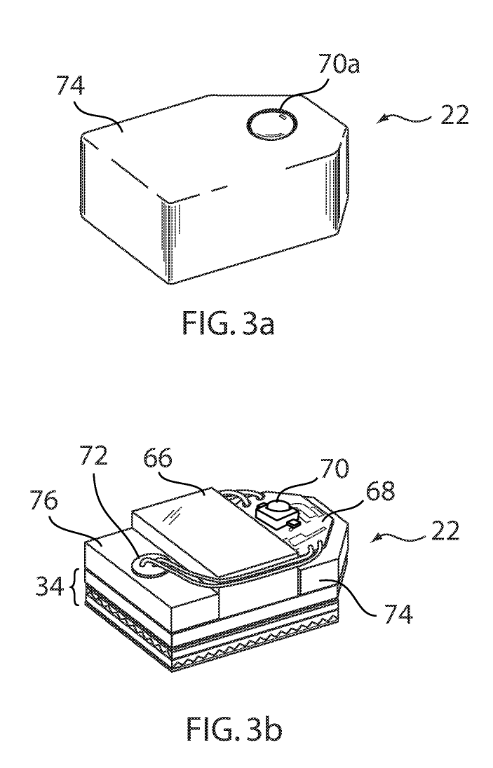

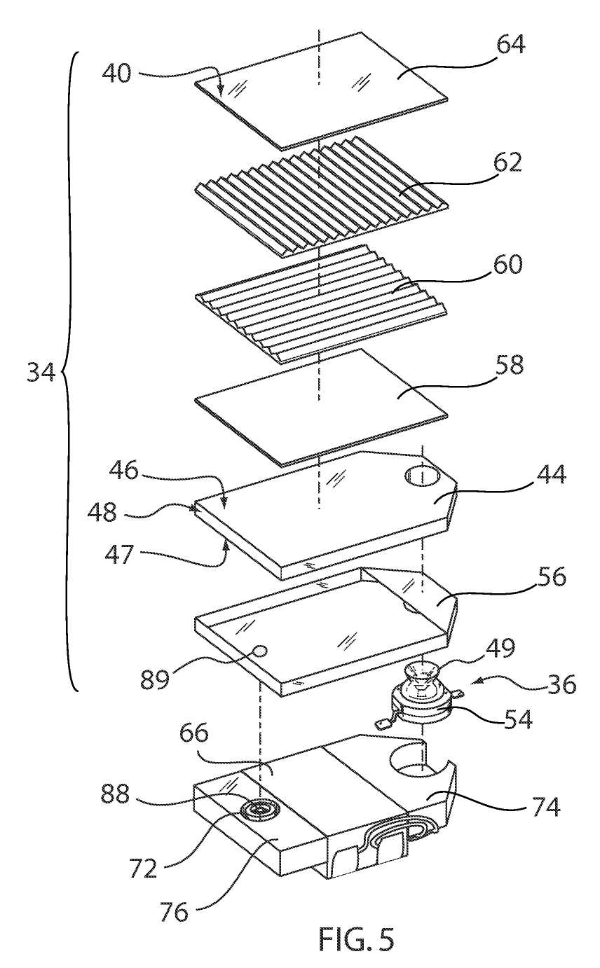

In one embodiment, FIGS. 3a-6b, optical device 22 comprises a diffuser assembly 34. Details of diffuser assembly 34 are shown in FIG. 5. Diffuser assembly 34 includes a light source 36 generating source light 38 and an emission surface 40 emitting emitted light 42. Light source 36 is preferably a light emitting diode (LED). The wavelengths of source light 38 that are used to activate photoactive compounds depend on the type of phototherapy being delivered. During photodynamic therapy using porphyrins such as photosensitizer protoporphyrin IX, the wavelengths used to activate this compound are typically violet (near 405 nm) or red (near 633 nm) and coincide with relative absorption spectrum maximums of the compound therefore activating them efficiently. PHOTOFRIN® is another photosensitizer that is typically activated using light with a wavelength near 632 nm, pyropheophorbides such as HPPH is activated near 665 nm, PC4 is activated near 668 nm, VISUDYNE® is activated near 690 nm, purpurinimides near 700 nm, and bacterio-purpurinimides near 800 nm. Many photoactive compounds absorb not only at their maxima, but in ranges around the relative absorption maxima. For the majority of phototherapy modalities, the wavelengths of source light 38 will be in the tissue optics window between near ultraviolet and near infrared, commonly within the ranges of at least one from the group including 400-410 nm, 630-635 nm, 660-700 nm, and 790-810 nm.

Light source 36 is coupled to at least one sheet waveguide 44, but may be a plurality of sheet wave guides. Each sheet waveguide 44 has a top sheet surface 46, a bottom sheet surface 47 and edge 48. Sheet waveguides generally comprise three layers of materials where the middle layer has an index of refraction that is greater than that of the adjacent layers. The thickness of the middle layer is substantially constant and can therefore confine some light within the middle layer through total internal reflection. Sheet waveguides may include a single planar sheet of material such as plastic in air, may be rigid or flexible, have thicknesses ranging from a few microns to several millimeters or more, and may guide light very strongly or allow a portion of the light to escape.

Diffuser assembly 34 may include a lens 49 to aid in the coupling of source light 38 into the edge of sheet waveguides 44. In general lens 49 is used to efficiently couple source light 38 into sheet waveguide 44 such that the etendue of the combination of light source 38 and lens 49 is matched to the etendue of the sheet waveguide. There are a variety of other structures for coupling light into and out of a sheet waveguide, such as prism couplers, lenses, microlens arrays, optical fiber butt coupling and other structures known in the art. A plurality of light extraction features 50 exist on at least one of either top sheet surface 46 or bottom sheet surface 47. Extraction feature 50 may be reflecting features or scattering features. Coupled light 41 is directed out of sheet waveguide 44 by extraction features 50 to become extracted light 43. Coupled light 41 may come from directing source light 38 from light source 36 into sheet waveguide 44, light re-emitted from treatment region 24 that is scattered into sheet waveguide 44 by extraction features 50 or extracted light 43 that is scattered into sheet waveguide 44 by extraction features 50. Diffuser assembly 34 may include additional components such as diode mount 54 for supporting light source 36. Diffuser assembly 34 may include a reflective layer 56 to help direct all light towards emission surface 40. Diffuser assembly 34 may include a diffusion layer 58 to enhance display uniformity. Diffusion assembly 34 may also include one or more brightness enhancing films to enhance emission brightness. Having a first brightness enhancing film 60 orthogonally oriented to a second brightness enhancing film 62, can aid in directing light towards emission surface 40. Diffusion assembly 34 may also include a protective cover 64 at emission surface 40. In many phototherapy modalities it is advantageous to have a large radiant emittance from device 22 beyond that which is generally available by backlights known in the art. In general, the emitted light will have a therapeutic radiant emittance in order to generate a desired therapeutic effect and achieve a desired light protocol.

The aforementioned combination of a LED, lens 49, and sheet waveguides 44 allows a radiant emittance above 62.8 mW/cm². It may also be an advantage to have an emission surface 40 that is flexible and can conform to the shape of the treatment region. This can be accomplished in the current embodiment by using a flexible diffuser assembly 34 that includes flexible components as is know in the art.

In an alternative embodiment, sheet waveguide 44 can be replaced with a light conversion waveguide. The conversion waveguide can be a sheet waveguide including a conversion material with fluorophores, semiconductor nanoparticles or other conversion material embedded therein or deposited thereon. Coupled light 41 will then be absorbed by the conversion material and converted light will be emitted isotropically to have a treatment wavelength that has a different wavelength than source light 38. A portion of the converted light will not be coupled in the waveguide due to the light's emission direction and will become extracted light 43. The distribution of emitted light 42 from the emission surface 40 can be controlled by the distribution and quantity of conversion material within or upon the conversion waveguide.

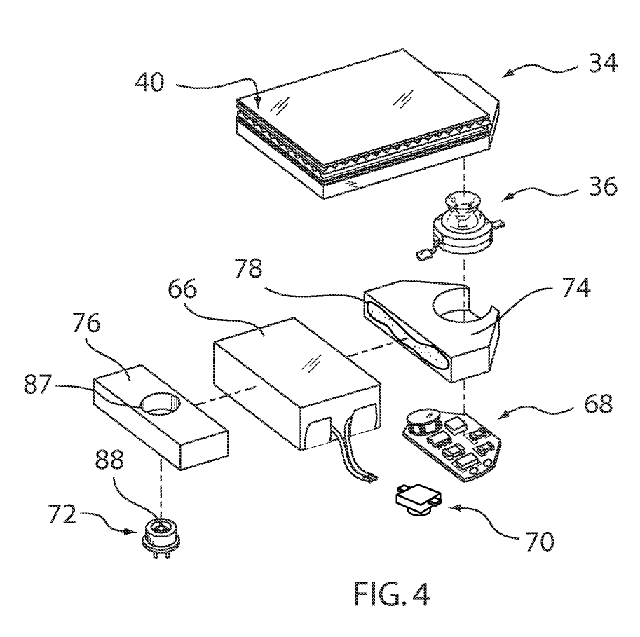

FIG. 4 shows additional components comprised in optical device 22. Power source 66 is preferably a lithium polymer battery or similar compact power source. Power source 66 not only powers power light source 36, but also acts a heat sink to aid in the dissipation of heat generated by the power source. Heat generated in optical device 22 can be transferred to treatment region 24 and non-treatment region 28 when optical device 22 is placed in close proximity to these regions. Casey et al. have shown that temperature over forty-three degrees Celsius can cause pain and tissue damage, the entirety of this reference is hereby incorporated by reference. Therefore it is critical to have good thermal control.

Optical device 22 also comprises a driver circuit 68. Driver circuit 68 works together with switch 70 and an optional feedback component 72 to regulate the duration and intensity of source light 38 generated by light source 36. Measuring the fluence rate provided to treatment region 24 by detector 88 enables delivery of a desired light delivery protocol to treatment surface 32 using feedback from a feedback loop. Switch 70 may have a tactile switch cover 70a that is integrated with case 77. Case 77 contains the components of optical device 22. A light source mount 74 and feedback component mount 76 each support their respective components. Source mount 74 and feedback component mount 76 also integrate via thermal paste 78 to power source 66 to aid in dissipating heat away from emission surface 40.

An important feature of phototherapy system 20 is to contain emitted light 42 to only treatment region 24. This is done by having case 77 and tactile switch cover 70a largely non-transparent to wavelengths emitted by source light 38. A portion of case 77 extends to emission surface 40. Packaged optical device 22 is substantially occlusive to exogenous light that may be incident on the optical device. Exogenous light can produce an unwanted phototherapeutic effect in treatment region 24. Exogenous light can be created by room lighting, the sun, or other light sources. Therefore, the packaged optical device 22 serves two purposes. First, case 77 prevents an undesired therapeutic effect in treatment region 24 from exogenous light. Second, case 77 prevents substantial amounts of emitted light 44 from exiting optical device 22 from regions other than emission surface 40 and potentially impinging on tissue regions or instrumentation outside of treatment region 24. Case 77 may additionally include a surrounding protective layer of transparent thermoplastic to prevent contamination of optical device 22.

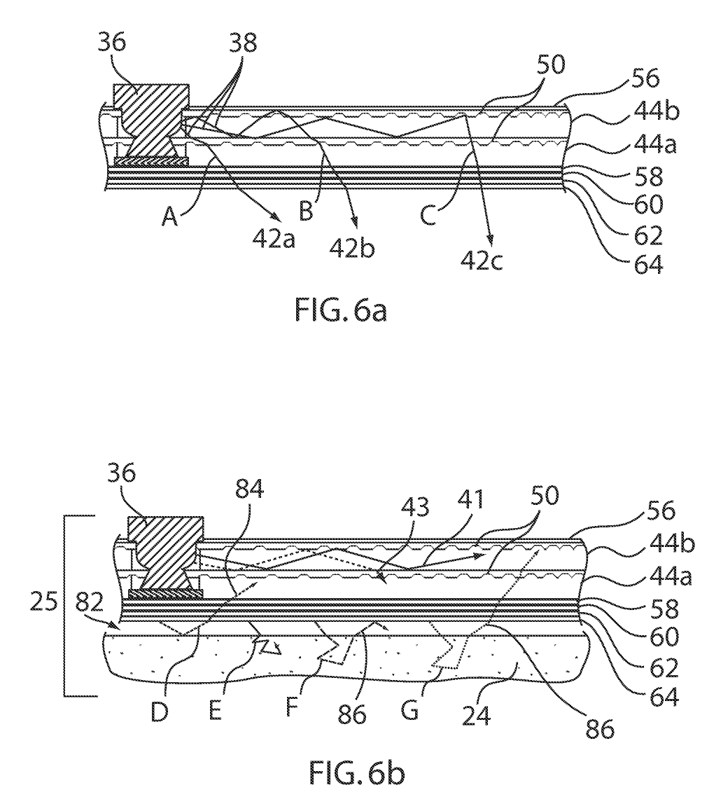

FIG. 6a shows how source light 38 interacts with components of diffusion assembly 34 to generate emitted light 42 exiting emission surface 40. This embodiment includes a first sheet waveguide 44a adjacent to a second sheet waveguide 44b. Both sheet waveguides 44a and 44b contain extraction features 50 to direct light out of the sheet waveguides. Reflective layer 56 prevents light in general from exiting device 22 in directions other than emission surface 40. In a potential light path A, source light 38 travels through all optical layers without being reflected and out emission surface 40 as emitted light 42A. In a potential path B, source light 38 travels along second sheet waveguide 44b until the source light escapes the second sheet waveguide and is reflected off of reflective layer 56 and then gets directed out emission surface 40 as emitted light 42B. In a potential path C, source light 38 travels along second sheet waveguide 44b until the light impinges an extraction feature 50 where the light then exits the second sheet waveguide and exits emission surface 40 as emitted light 42C. These illustrative paths and a multitude of other potential light paths combine to create the total output of emitted light 42 from emission surface 40.

FIG. 6b shows an embodiment in which optical device 22 is in proximity to treatment region 24. In a potential path D, emitted light 42 exits emission surface 40, travels through air layer 82, is reflected by treatment surface 32 and then re-enters optical device 22 as reflected light 84. In alternative embodiments, air layer 82 is filled with an index matching fluid, a pharmaceutical compound, or other materials known in the art of phototherapy. In a potential path E, emitted light 42 exits emission surface 40 and enters treatment region 24 where the emitted light is absorbed by a photoactive compound, such as a photosensitizer. Alternatively, emitted light 42 could be absorbed by a chromophore, a bacterium, or other absorptive material. In a potential path F, emitted light 42 exits emission surface 40 and enters treatment region 24 where the emitted light is scattered and re-emitted from treatment surface 32 as scattered light 86 in the direction of optical device 22. Scattered light 86 is then reflected from protective cover 64 back in the direction of treatment region 24. In a potential path G, emitted light 42 exits emission surface 40 and enters treatment region 24 before being emitted from the treatment region as scattered light 86 in the direction of optical device 22. Reflected light 84 is transmitted through protective cover 64, through brightness enhancing films 60 and 62, through diffusion layer 58, through first sheet waveguide 44a and enters second sheet waveguide 44b. There are many possible paths that light can travelin phototherapy system 20 when that system includes optical device 22 in proximity to treatment region 24.

Optical device 22 and treatment region 24 create a resonator 25 in which a portion of source light 38 is transferred from optical device 22 to treatment region 24 and back to optical device 22 multiple times, resulting in the buildup of source light 38 within resonator 25. The buildup of source light 38 resulting from this optical resonance and the magnitude thereof, will generally not be similar when optical device 22 is in operation in free space versus when the optical device is in proximity to treatment region 24. In general, the magnitude of the buildup of source light 38 will depend on the likelihood that emitted light 42 will be re-emitted by treatment region 24 in the direction of optical device 22 and the likelihood that the re-emitted light will be reflected or scattered from the optical device back in the direction of the treatment region. By coupling light source 36 with treatment region 24 so that the source light 38 in combination with reflected light 84 and scattered light 86 from the treatment region, the combined radiant emittance of the system can be greater than 1.5 times the radiant emittance of just the source.

During operation of device 22 in free space, the radiant emittance from emission surface 40 is comprised of a large number of source light 38 photons that represent a variety of paths through the device 22. During the delivery of phototherapy to treatment region 24, however, the radiant emittance from emission surface 40 is comprised of source light 38 photons in addition to photons from scattered light 86 and reflected light 84. The result is that the radiant emittance during the delivery of phototherapy exceeds the radiant emittance in free space. Optical device 22 may have a reflective layer 56 which can enhance this affect. Using optical scattering, absorption and index of refraction parameters in the normal range of tissue, the radiant emittance when the device 22 is in near contact can exceed 1.5 times the radiant emittance of when the device is in free space. This increase in radiant emittance therefore allows a decrease in source light 38 to obtain the same radiant emittance obtained in free space. This is critical for reducing the power consumption of optical device 22. Moreover, the contribution to the radiant emittance from scattered light 86 and reflected light 84 is dependent on the optical properties of the treatment region 24. Since optical properties of a treatment region 24 can vary significantly by tissue type and person to person, it may be important to control light delivery with a feedback component 72. Similarly, during operation of optical device 22 in free space, free-space emission pattern 130 from emission surface 40 results from a large number of source light 38 photons that represent a variety of paths through the optical device 22. During the delivery of phototherapy to treatment region 24, however, proximity emission pattern 142 results from source light 38 photons in addition to photons from scattered light 86 and reflected light 84. The result is that proximity emission pattern 142 is different from that of free-space emission pattern 130 measured.

The buildup of source light 38 in resonator 25 comprising optical device 22 and treatment region 24 is increased as the amount of phototherapy device backscattering and reflectivity increases. The optical device re-emission percentage is the chance that source light 38 that is re-emitted by treatment region 24 will impinge on emission surface 40 of the optical device 22 and will then be re-emitted by the optical device in the direction of treatment region 24, whether due to scattering, diffuse or specular reflection, or other means. Similarly optical device loss percentage is the chance that source light 38 that is re-emitted by treatment region 24 and that impinges on emission surface 40 of the optical device 22 and is then absorbed, directed away from the treatment region or otherwise removed from contributing to the buildup of source light 38 within resonator 25. For representative tissue scattering/absorption coefficients of 100 cm⁻¹/5 cm⁻¹, a phototherapy device with a re-emission percentage of greater than 90% may result in an approximate 50% increase in fluence rate to the target region through the buildup of source light 38, representing an irradiance on the treatment surface that can be greater than 1.5 times the initial radiant emittance and generating a desired fluence rate from said source light upon the treatment surface. One advantage of this increase in fluence rate is that it increases the maximum fluence rate to treatment region 24 for light source 36. This in turn provides phototherapy with a higher fluence rate than could be accomplished with a system having a lower re-emission percentage. It is an advantage of this fluence rate buildup that results in reducing electrical power needs of light source 36, improved device thermal management and a smaller physical space.

In one embodiment optical device 22 further includes components that allow optical measurements to be collected and used to provide a measurement of fluence and fluence rate for light delivered to treatment region 24. These measurements are further used as a source of feedback control to optical device 22. These measurements are collected by detector 88 that is part of feedback component 72. Detector 88 may be a silicon photodiode or similar detector element, positioned within feedback aperture 87 of feedback component mount 76. In one embodiment, reflective layer 56 includes a transmissive region 89 that is a thinned region of the reflective layer, partially transparent portion of the reflective layer, a pinhole aperture or other effective structure. Detector 88 may comprise at least one from the group including a linear array of detectors, a detector which is filtered to respond largely to only the emission wavelength of source light 38, and a detector which largely responds to emission from fluorophores (such as photosensitizers) in treatment region 24, or other similar detectors. Detector 88 may be integrated with power source 66 and driver circuit 68. Measurements from feedback component 72 may be used in conjunction with driver circuit 74 to provide dynamic optical feedback, whereby the total fluence is calculated and parameters of light source 36 are controlled. As an illustrative example, detector 88 may measure a relative amount of light from light source 36 incident thereon. Data from detector 88 is then used to drive a feedback circuit of driver circuit 68 that in turn modifies the output of light source 36. Feedback component 72 thereby can provide accurate delivery of light to treatment region 24, incorporating the aforementioned optical feedback effects including any of a measure of light source power, a measure of fluence rate provided to treatment region 24, a measure of total fluence provided to the treatment region, a measure of fluorescence from the treatment region, a measure of reflection from the treatment region, a measure of scattering from the treatment region, and a measure of the location of the treatment region or chromophore concentrations within the treatment region. In the preferred embodiment, detector 88 is a calibrated photovoltaic sensor adjacent to transmissive region 89 of the diffuser assembly's reflective layer 56. This allows detector 89 to sample a small amount of source light 38 in diffuser assembly 34 and provide feedback to light source 36 to either increase or decrease output in order to deliver a prescribed fluence rate to treatment region 24.

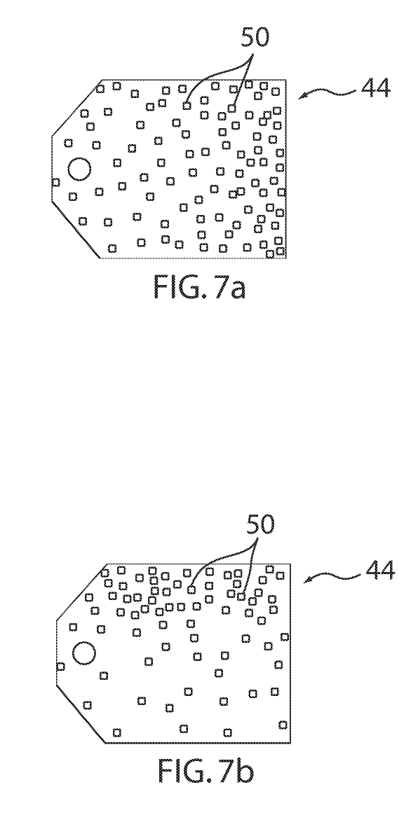

FIG. 7a illustrates another important feature of optical device 22 where sheet waveguide 44 has a distribution of extraction features 50. Extraction features 50 are used to both direct light into and direct light out of sheet waveguide 44. As is know in the art of backlighting, extraction features 50 may comprise paint dots or molded total internal reflection bumps. In one embodiment, the distribution of extraction features 50 is prescribed using a non-sequential ray tracing model that incorporates both the optical parameters of optical device 22 and optical parameters of treatment region 24. The optical parameters of treatment region 24 may include approximated or measured values for index of refraction, absorptivity, scattering and surface uniformity. These optical parameters may be homogenous or heterogeneous throughout treatment 24. In one embodiment, the features are prescribed so as to produce a uniform distribution of light impinging on treatment surface 32 when coupled with treatment region 24. FIG. 7b illustrates a top down view of sheet waveguide 44 having another possible distribution of extraction features 50.

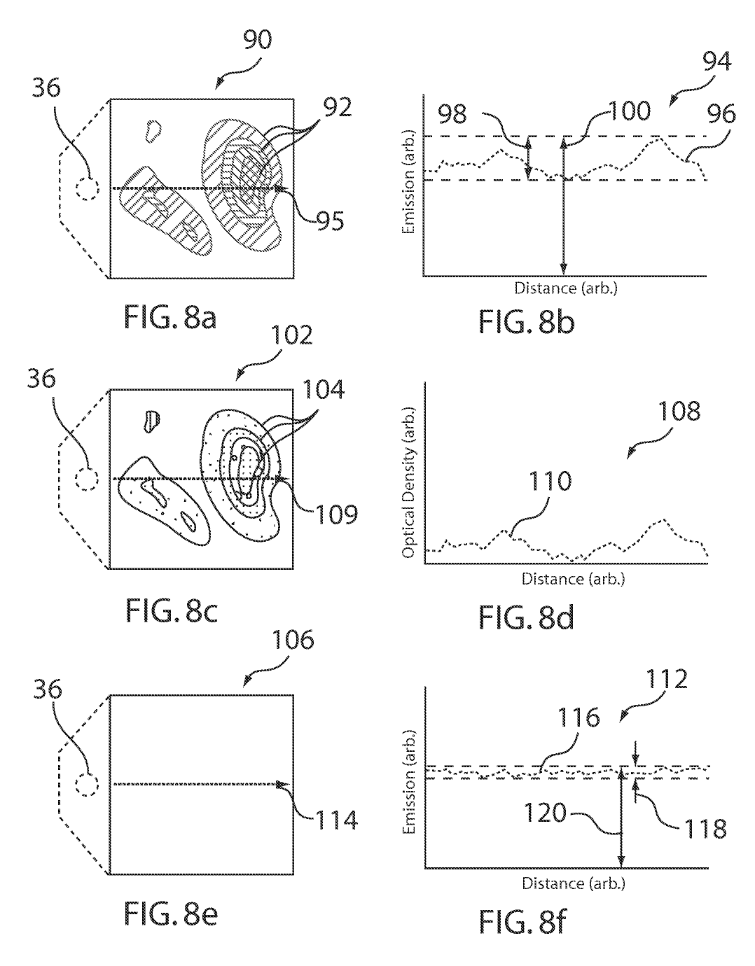

FIGS. 8a-f illustrate the effect of a heterogeneous layer in combination with optical device 22 to produce a desired emission pattern. The addition of a heterogeneous layer may be used to mitigate the effects of fabrication artifacts associated with light source 36 and other diffusion assembly components. In detail, FIG. 8a illustrates a first light emission pattern 90 that might be produced by optical device 22. Superimposed on first light emission pattern 90 are emission contours 92. FIG. 8b further illustrates a first line scan emission plot 94 oriented along a first measurement region 95 (the median) of first light emission pattern 90. First line scan emission plot 94 starts proximal to light source 36 and extends away from the light source. The variation of the first emission scan magnitude 96 can be expressed as the ratio of the magnitude of the range of emission 98 scan to the magnitude of the maximum first emission 100. In certain phototherapy applications, it is desirous to minimize emission variation in order to provide even treatment and provide a substantially homogenous light emission pattern. FIG. 8c illustrates a heterogeneous absorptive layer 102 with superimposed optical density contours 104. When absorptive layer 102 is used in combination with optical device 22 having first light emission pattern 90 shown in FIG. 8a, the combination produces second light emission pattern 106. Absorptive layer 102 could be a transparent plastic sheet with paint dots thereon, a laminate of shaped neutral density filters, or other material as is known in the art. FIG. 8d further illustrates a first line scan optical density plot 108 oriented along second measurement region 109 (the median) of absorptive layer 102. First line scan optical density plot 108 starts proximal to light source 36 and extends away from the light source. In the preferred embodiment, optical density scan 110 is substantially correlated to emission scan 96. In combination, optical device 22 and absorptive layer 102 result in a second light emission pattern 106, FIG. 8e. FIG. 8f further illustrates a second line scan emission plot 112 for a third measurement region 114 (the median) of second light emission region 106. Second line scan emission plot 112 starts proximal to light source 36 and extends away from the light source. The variation of second emission scan 116 can similarly be expressed as the ratio of the magnitude of the range of the measurement 118 to the magnitude of the maximum second emission 120. In this embodiment, the second emission variation is less than the first emission variation. The reduction in variation could be similar across the entire emission pattern from the combination of optical device 22 and absorptive layer 102. The parameters of the absorptive layer 102 may be determined by imaging the first light emission pattern 90 of optical device 22. A heterogeneous reflective layer or scattering layer could be used in a similar way to that of absorptive layer 102. Optical resonance, as describe previously, may be considered in the design of the optical density pattern of absorptive layer 102. The optical density pattern may be determined through modeling which incorporates the parameters of optical device 22 and treatment region 24.

As previously mentioned many phototherapy modalities benefit from a uniform, or otherwise predicable, light distribution on treatment region 24. This patent application therefore includes a method of delivering phototherapy to treatment region 24. The method includes the steps of providing optical parameters of the treatment region such as absorption, scattering, tissue geometry and the index of refraction of the treatment region. These parameters can, for example, be estimated by skin type or can be explicitly measured as is known in the art of biomedical optics. These parameters can be heterogeneous throughout treatment region 24 or can be approximated as homogenous. In addition, this method provides a desired light distribution over treatment region 24, which could be a uniform distribution or could be a distribution in which more light is incident on a preferred area, such as a pigmented lesion. This method also provides light source 36 with a set of optical parameters. These parameters may include wavelength, radiance, dimensions, or other relevant parameters. Using the optical parameters of both the light and the tissue, one can model a distribution of light extraction features that would provide a desired light distribution on treatment region 24. For example, Monte Carlo (MC) modeling of light propagation can be performed to trace the likely paths of photons provided tissue optical parameters, source optical parameters and a distribution of light extraction features. This is analogous to the process for modeling light extraction features in a backlight (Optical Research Associates). Following the modeling of extraction features, optical device 22 can be constructed using the modeled distribution of light extraction features. The appropriate distribution of light extraction features can be accomplished, for example, using paint dots or embossed scattering features on waveguide as described by Optical Research Associates. Once optical device 22 is built, phototherapy is delivered with the appropriate light distribution to treatment region 24.

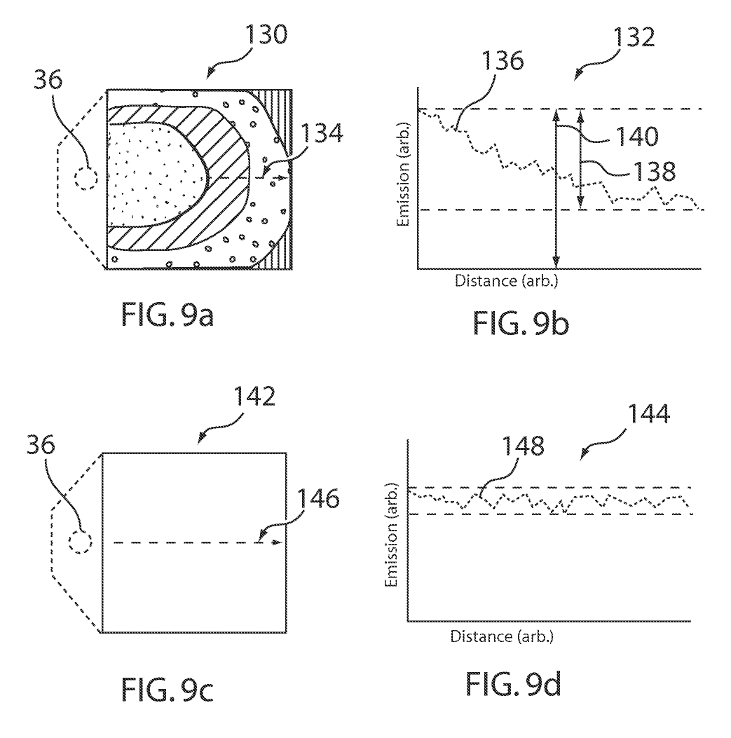

As mentioned above, many phototherapy modalities benefit from a uniform, or otherwise predicable, light distribution on treatment region 24. FIGS. 9a-d therefore illustrate how the desired emission from optical device 22 in free space may not be uniform but can provide uniform irradiance to treatment region 24 when in near contact. This results, as before, from the effect of resonance in the form of reflected light 84 and scattered light 84. FIGS. 9a and 9b show an illustrative free-space emission pattern 130 from an embodiment of optical device 22 when the optical device is not in proximity to the treatment region 24. Free-space emission pattern 130 is therefore not subject to the aforementioned optical resonance. An illustrative third line scan emission plot 132 is shown for a free-space measurement region 134 that is oriented along the median of free-space emission pattern 130, starting proximal to the light source 36 going toward the distal end. The variation of the free-space emission line scan 136 can be expressed as the ratio of the magnitude of the range 138 of the scan to the free-space maximum 140 of the scan. FIG. 9c and FIG. 9d similarly show an illustrative proximity emission pattern 142 from optical device 22 when in proximity to the treatment region 24 and therefore subject to optical resonance from the tissue and which may result from such effects as reflection and scattering. An illustrative proximity emission line scan plot 144 is shown for proximity measurement region 146. The variation of the resulting proximity emission scan 148 is less than that of free-space emission line scan 136. The variation over the entire emission pattern could also be reduced similarly.

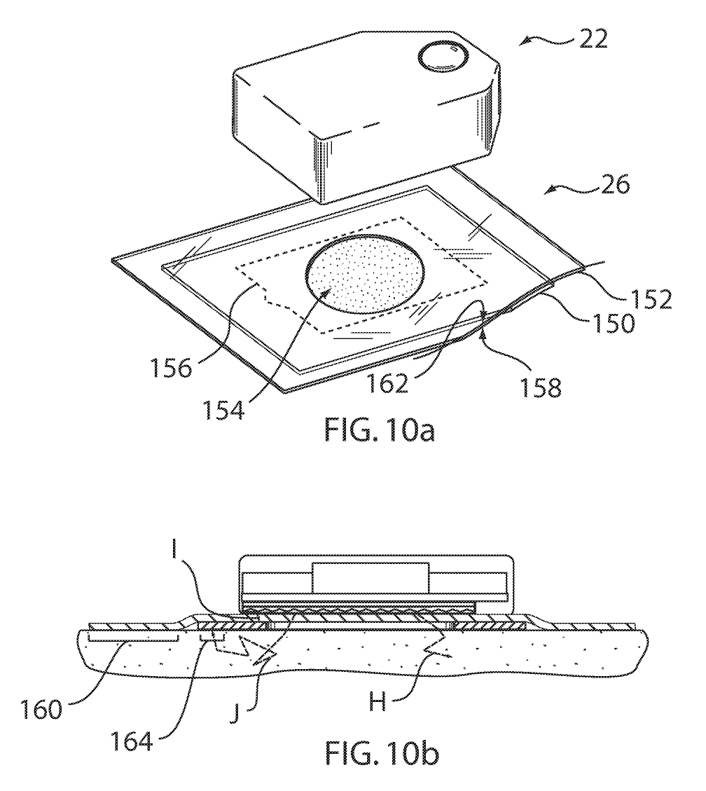

FIG. 10a illustrates an embodiment wherein phototherapy system 20 further comprises a treatment patch 26 adjacent to treatment region 24. Treatment patch 26 provides a convenient way to orient and position optical device 22. Treatment patch 26 also provides an added way to control the therapeutic light distribution to only treatment region 24. In one embodiment, treatment patch 26 includes a blocking layer 150 and a transparent adhesive layer 152. Blocking layer 150 is made from a material which substantially and preferentially occludes light with wavelengths of emitted light 42. Blocking layer 150 is sized so that it extends slightly beyond the edges of emission surface 40. Blocking layer 150 has a treatment aperture 154 therethrough. In one embodiment, an alignment mark 156 is printed on blocking layer 150. The transparent adhesive layer 152 extends beyond the edges of blocking layer 150 and has an adhesive surface on a bottom side 158. An adhesive layer portion 160 is in contact with blocking layer 150. Treatment patch 26 may alternatively include a single blocking layer with adhesive surfaces. In an alternative embodiment, optical device 22 could be replaced by an illuminating layer comprising organic light emitting diodes, an electroluminescent film, a fiber device as described by Parker, or other light producing layer.

It has been shown that temperature over forty-three degrees Celsius can cause pain and tissue damage (Casey et al.). Therefore it is critical to have good thermal control in order to prevent heat generated in optical device 22 from being transferred to treatment region 24 and non-treatment region 28 when optical device 22 is placed in close proximity to these regions. To ensure not having the temperature of treatment region go above forty-three degrees Celsius, it may be advantageous to include an endothermic layer. This endothermic layer may reside in either the treatment patch between the optical device and the treatment region or in the optical device near the light source where most of the heat is generated. An appropriate endothermic layer for the patch could comprise a cold pack thermally coupled to blocking layer 152. The cold pack could for example include a thermally conductive plastic. The cold pack could further comprise a number of endothermic processes or reactions such as evaporative cooling, dissolution of ammonium chloride in water, or other known reactions and processes. Preferably the cold pack would be activated for the entire duration of the therapy as heat is generated and keep the temperature of treatment region 24 below forty-three degrees Celsius. Similarly, an appropriate endothermic layer within the device 22 could be a heat sink material such as copper which is thermally coupled to a cold pack as described above.

During the application of treatment patch 26 to treatment region 24 and non-treatment region 28, the treatment patch is positioned such that the treatment region is circumscribed by treatment aperture 154. In a preferred embodiment, treatment patch 26 is largely translucent such that the features of treatment region 24 and non-treatment region 28 are substantially visible to the eye when viewed through the laminate, this aids in proper placement of the laminate. Treatment patch 26 is placed in contact with treatment region 24 and non-treatment region 28 so that an adhesive layer portion 160 is in contact with non-treatment region 28 providing a way to attach the treatment patch to the treatment region and the non-treatment region. Optical device 22 is subsequently positioned using alignment mark 154 as a guide, ensuring proper position and orientation. In one embodiment, optical device 22 adheres to adhesive layer 152 through adhesive material on a top side 162 of the adhesive layer.

During phototherapy, as illustrated in FIG. 10b, emitted light 42 may follow a potential light path H exiting optical device 22 and being substantially transmitted through transparent adhesive layer 152, this light is then transmitted through treatment aperture 154 and impinges on treatment region 24 producing a phototherapeutic effect. In a potential light path I, emitted light 42 from optical device 22 impinges on blocking layer 150 wherein this light is largely absorbed. In a potential light path J, emitted light 42 is transmitted through transparent adhesive layer 152, is scattered within treatment region 24 and then impinges onto blocking layer 150 wherein that light is largely absorbed. Hence, the distribution of light within the treatment region 24 is substantially influenced by blocking layer 150 and treatment aperture 154. Similarly, a blocking layer portion 164, which extends beyond the edge of the emission surface 40, limits reflected light 84 and scattered light 86 from exiting the treatment patch 26. In some treatment protocols it is important to prevent light from being emitted from the phototherapy system as it can provide unintended phototherapy to other regions or represent an eye hazard. It is also known in the art that blocking layer 150 could comprise a partially absorbing film or a selectively reflective film, such as a band-reject filter.

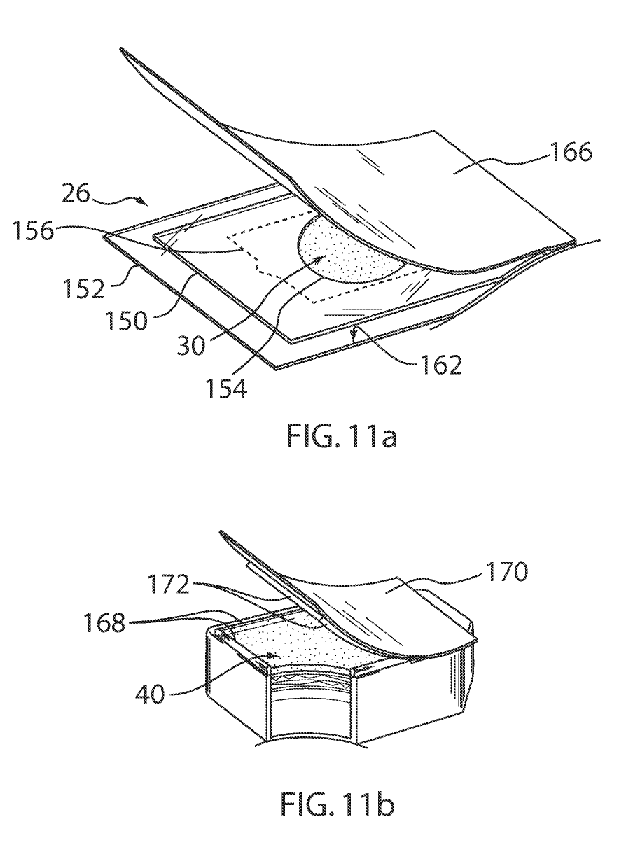

FIG. 11a shows an embodiment where treatment patch 26 further comprises a therapeutic compound 30. Therapeutic compound 30 substantially fills treatment aperture 154 of blocking layer 150. This embodiment further comprises a removable film 166 that is used to both enclose therapeutic compound 30 within treatment aperture 154 and protect the adhesive layer 152. During application, removable film 166 may be peeled away from treatment patch 26 and treatment compound 30. Treatment patch 26 is then positioned above the treatment region 24 as outlined above. This process places treatment compound 30 in contact with treatment region 24. Optical device 22 is then positioned with respect to treatment patch 26 as previously described. Removable film 166 may be largely non-transparent and may consist of a metal foil. Following phototherapy treatment, optical device 22 is removed and removable film 166 may be adhered to the top side 162 of treatment patch 26 to form an occlusive layer to treatment region 24. This occlusive layer may serve to prevent further phototherapy from exogenous sources.

FIG. 11b shows an embodiment for including therapeutic compound 30 with optical device 22 for convenient delivery to treatment region 24. In this embodiment, compound 30 substantially fills a depression provided by the combination of plastic ridges 168 and front emission surface 40. A removable interlocking film 170 has ridges with cooperative depressions 172, which allow a reversible seal to be made between optical device 22 and interlocking film 170. This combination encapsulates therapeutic compound 30. Interlocking film 170 is removed prior to the application of optical device 22 to treatment patch 26 as described above. This causes therapeutic compound 30 to be in contact with treatment region 24 through treatment aperture 154.

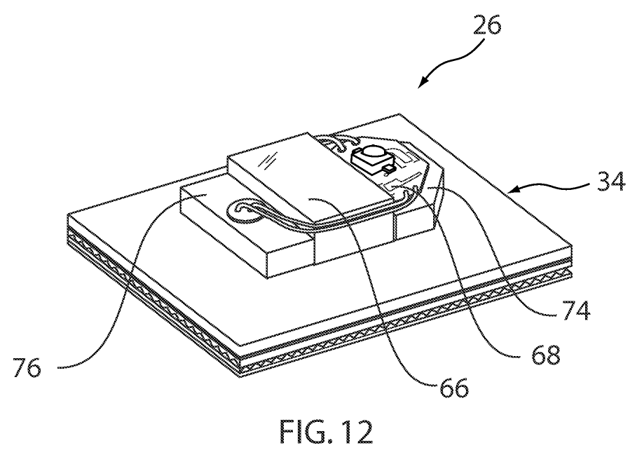

FIG. 12 shows an alternative embodiment of a optical device 26 where diffuser assembly 34 extends beyond the footprint of light source mount 74, feedback mount 76, power source 66 and driver circuit 68.

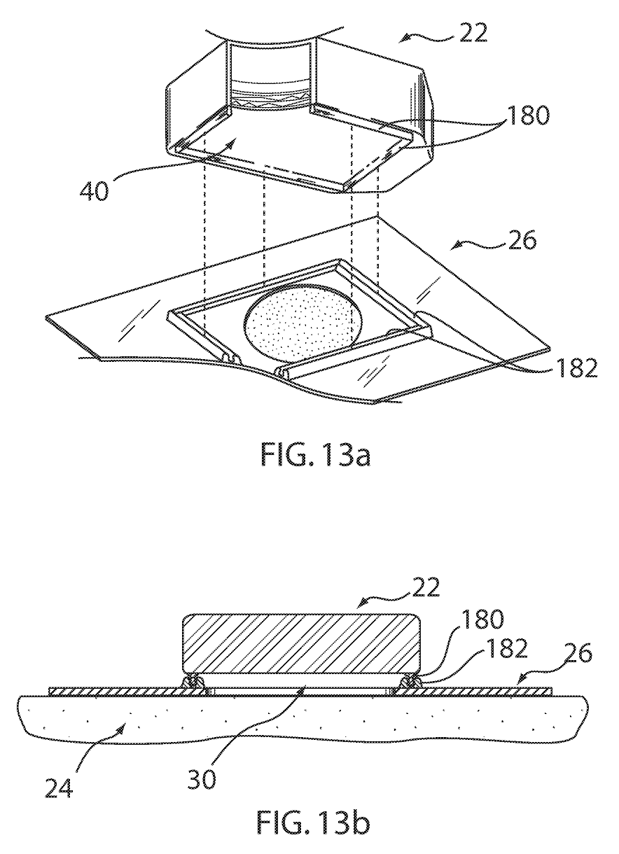

FIG. 13a illustrates an alternative embodiment for securing an optical device 22 to treatment patch 26 using interlocking members. In this embodiment, a first set of plastic ridges 180 are positioned on the edge of emission surface 40 of optical device 22. Ridges 180 are shaped such that they correspond to, and interlock with, a second set of ridges with cooperative depressions 182 when placed in contact. In some embodiments the interlocked members can be disconnected easily, meaning with relatively little separating force, and whereas in other embodiments the interlocking is substantially permanent.

As illustrated in FIG. 13b, in one embodiment of attachment using interlocking members, a gap 184 may be created between treatment region 24 and emission surface 40. This gap may be filled with a phototherapy compound 30, such as a photosensitizer compound, or may be filled with thermally insulating material to limit thermal conduction between the optical device 22 and treatment region 24.

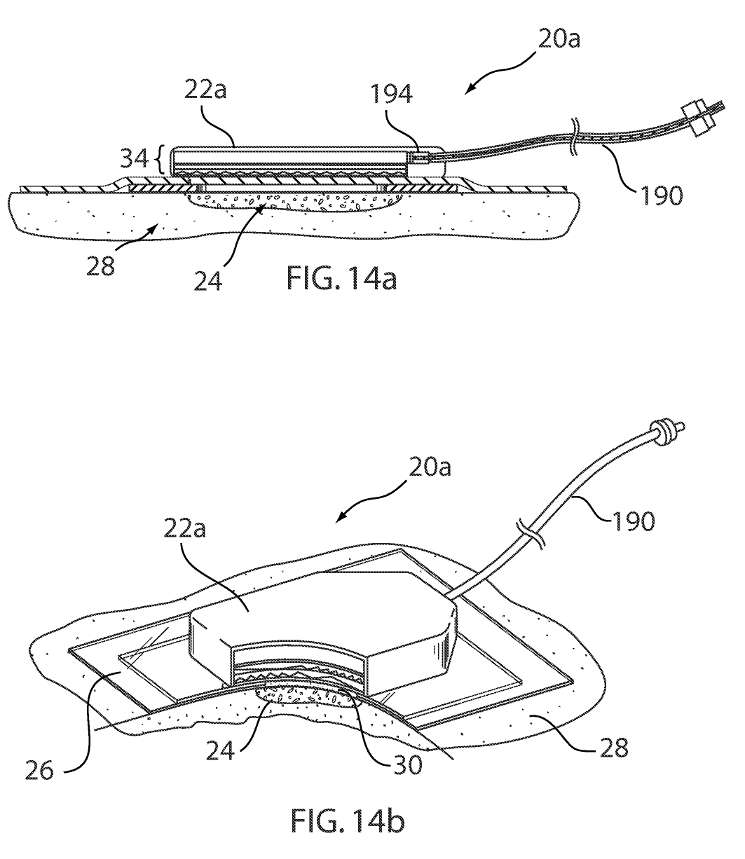

FIGS. 14a and 14b show an alternative embodiment of optical device 22 where light source 36 and the light source's associated power supply have been replaced with by a remote light source such as a laser source (not shown) that is tethered to the device by an optical waveguide, such as an optical fiber 190. This optical device is a tethered optical device 22a. In this embodiment tethered phototherapy system 20a, comprises all of the elements described previously for phototherapy system 20 (diffuser assembly 34, feedback component 72, treatment patch 26 and treatment compound 30), however, those elements that are associated with generating light from within optical device 22 have been removed and replaced by connection to a remote light source. For example, power supply 66, switch 70 and light source 36 are no longer contained within optical device 22a. Instead optical fiber 190 is connected by a diffusion tip 194 to diffuser assembly 34. There are many viable embodiments of diffusion tip 194. The preferred embodiment comprises two layers perpendicular to the optical axis of the fiber tip and the plane of the treatment surface. The first layer is a thin film optical grating located that preferentially directs light in a fan distribution parallel to the plane of the treatment surface and the second layer is a diffusing layer, which homogenizes the distribution of light within the fan distribution. This structure is preferred because it allows an emulation of an LED light source and is therefore compatible with standard sheet waveguides using in backlighting applications. One of the main advantages to this type of tethered optical device 22a is that heat associated with generating light is now removed from the phototherapy system and thermal management of treatment region 24 can be better controlled.

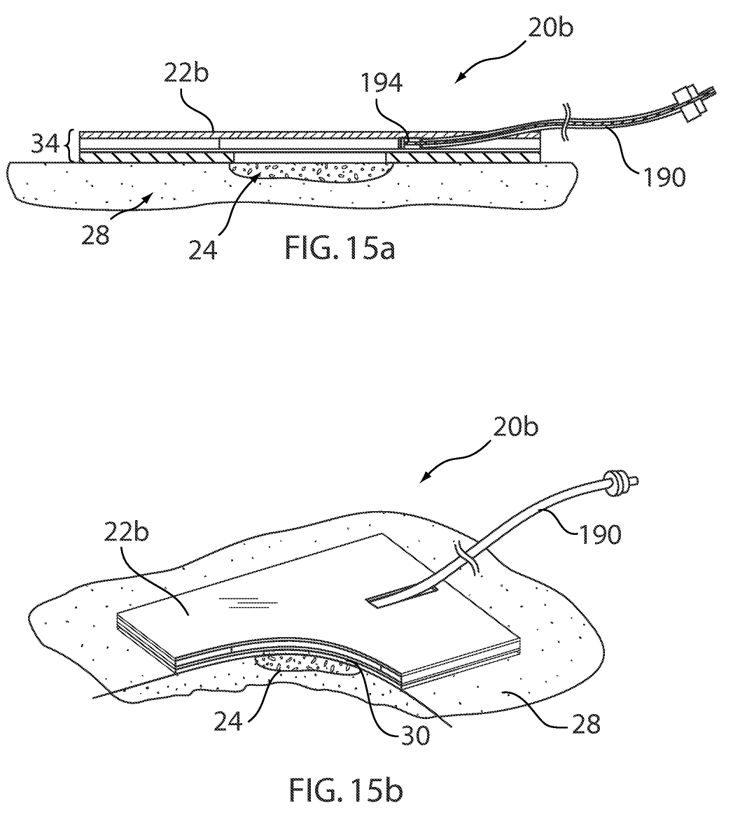

FIGS. 15a and 15b show an alternative embodiment of optical device 22 where light source 36 and the light source's associated power supply have been replaced with by a laser light source (not shown) that is tethered to the device by an optical fiber 190. This optical device is a thin film tethered optical device 22b. In this embodiment thin film tethered phototherapy system 20b, comprises all of the elements described previously for phototherapy system 20 (diffuser assembly 34, feedback component 72, treatment patch 26 and treatment compound 30), however, for those elements that are associated with generating light from within optical device 22 have been removed and replaced by connection to a remote light source. For example, power supply 66, switch 70 and light source 36 are no longer contained within optical device 22b. Instead optical fiber 190 is connected by diffusion tip 194 diffuser assembly 34. Furthermore, diffuser assembly 34 is now constructed as a thin film laminate. One of the main advantages to this type of optical device is that as described above the heat associated with generating light is now removed from the phototherapy system and thermal management of treatment region 24 can be better controlled. Another advantage is that this optical device system can be fabricated by way of a thin film reel to reel process.

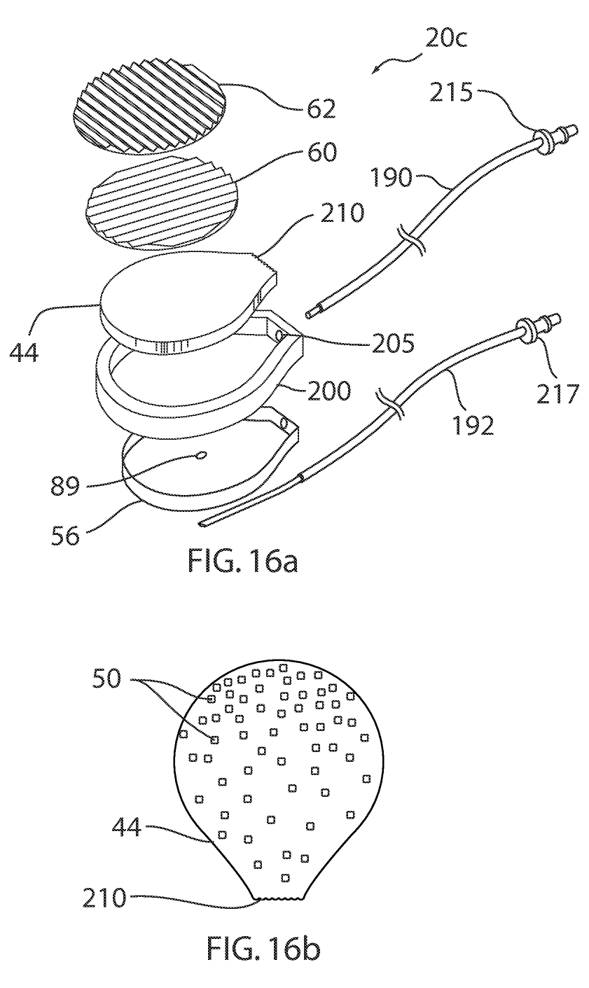

FIGS. 16a and 16b show an alternative embodiment of optical device 22 where diffuser assembly 34 is mounted with support structure 200. This optical device is supported tethered optical device 22c. In this embodiment supported tethered phototherapy system 20c, comprises all of the elements described previously for phototherapy system 20 (diffuser assembly 34, feedback component 72, treatment patch 26 and treatment compound 30), however, for those elements that are associated with generating light from within optical device 22 have been removed and replaced by connection to a remote light source. For example, power supply 66, switch 70 and light source 36 are no longer contained within optical device 22c. Instead optical fiber 190 is connected to support structure 200 by ferrule 205. Light exiting optical fiber 190 passes through lenticular source coupler 210 on an edge of sheet waveguide 44 with light extraction features 50. One advantage to this type of optical device is that support structure 200 can provide additional strength to the device and can be used to provide a prescribed curvature to diffuser assembly 34 that can be used to provide an emission surface that is conformal to a non-planar treatment region 24. Lenticular source coupler 210 can be a one-dimensional microlens array, optical grating or other optical features on the edge of sheet waveguide 44 which spreads incident light from optical fiber 190 in the plane of sheet waveguide 44 and increases the uniformity of the light distribution therein. Lenticular source coupler 210 can be formed by cutting into, molding a surface or adding material to sheet waveguide 44 as is known in the art of grating and microlens manufacturing. In this embodiment, optical fiber 190 is terminated on the distal end by snap coupler 215, which can be used to rapidly connect and disconnect device 22c to a complimentary source fiber (not shown).

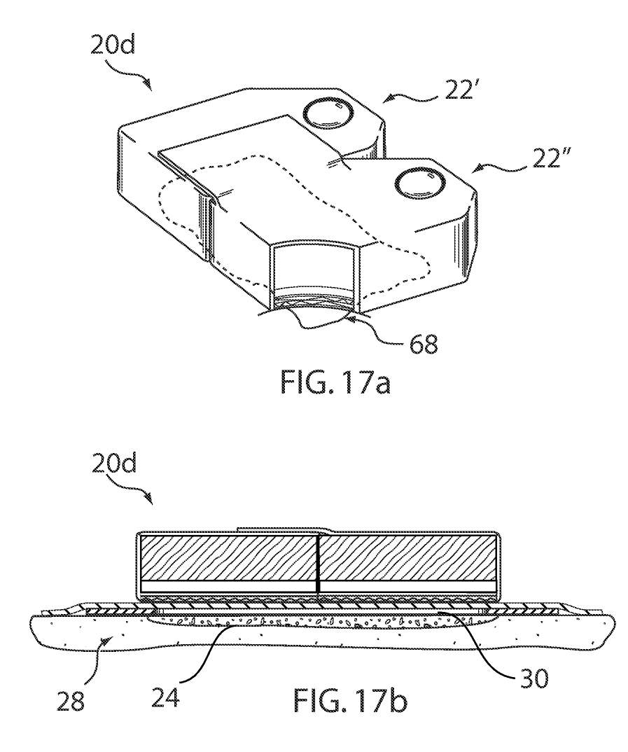

FIGS. 17a and 17b show an alternative embodiment of optical device 22 whereby a first optical device 22′ and a second optical device 22″ are used in combination to simultaneously provide phototherapy to a contiguous expanded treatment region 24. In this embodiment, each optical device (22′22″) has at least one geometrically cooperative matching edge. Optical devices (22′, 22″) are placed adjacent to each other over the treatment area to create a collective emission surface area that substantially matches the area of the treatment region. Each pair of matching edges produces a continuous distribution of light over the collective emission surface area, wherein emission region is larger than a single emission region. Geometric phototherapy system 20d uses optical devices (22′, 22″) in combination to provide phototherapy to a contiguous treatment region 24 of a variety of sizes and shapes.

The phototherapy system 20, 20a, 20b, 20c and 20d are all designed to provide a therapeutic affect that is at least one from the group including photodynamic therapy, photodynamic acne therapy, photodynamic psoriasis treatment, bacterial sterilization, wound healing, treatment of cutaneous T cell lymphoma, treatment of vitiligo, treatment of fungal infection, treatment of lichen planus, treatment of granuloma annulare, treatment of warts, treatment of pityriasis rosea, treatment of generalized itching from various causes, treatment of atopic and other types of eczema, treatment of non-melanoma skin cancer, photorejuvination, treatment of hidradenitis suppurativa, treatment of Lichen sclerosus, treatment of scleroderma, treatment of alopecia areata, treatment of lichen planus, treatment of Darter's disease, treatment of vulvar intraepithelial neoplasia, treatment of cervical intraepithelial neoplasia, treatment of penile intraepithelial neoplasia, treatment of anal carcinoma in situ, treatment of Barrett's esophagus, treatment of oral candidiasis, anti viral therapy, hair removal, treatment of Bowen's disease, treatment of oral cancer, treatment of sun damage, cosmetic skin improvement, treatment of oily skin, treatment of enlarged sebaceous glands, and treatment of wrinkles

While several embodiments of the invention, together with modifications thereof, have been described in detail herein and illustrated in the accompanying drawings, it will be evident that various further modifications are possible without departing from the scope of the invention. Nothing in the above specification is intended to limit the invention more narrowly than the appended claims. The examples given are intended only to be illustrative rather than exclusive.

What is claimed is:

1. A phototherapy system for a treatment region having a treatment surface, comprising:
an optical device including:
a) a light source generating source light;
b) a sheet waveguide, said sheet waveguide having a pair of sheet surfaces and an edge, said light source coupled to direct said source light into said sheet waveguide;
c) a plurality of light extraction features on at least one of each pair of sheet surfaces; and
d) an emission surface emitting emitted light, said emitted light having a wavelength to activate a photoactive compound, said emitted light having a therapeutic radiant emittance.

2. A phototherapy system as recited in claim 1, wherein said optical device in combination with the treatment region forms a resonator.

3. A phototherapy system as recited in claim 1, wherein said optical device further includes a brightness enhancing film.

4. A phototherapy system as recited in claim 1, wherein the optical device further includes a lenticular source coupler that distributes light from the light source into the sheet waveguide, wherein said light source is an optical fiber.

5. A phototherapy system as recited in claim 1, wherein said optical device further includes a light feedback component that measures light within said optical device and provides feedback to said light source.

6. A phototherapy system as recited in claim 5, wherein said optical device provides a prescribed light delivery protocol for the treatment region.

7. A phototherapy system as recited in claims 1, wherein said optical device further includes a heterogeneous absorptive layer that reduces spatial variations in said scattered light and said reflected light from the treatment region.

8. A phototherapy system as recited in claim 1, whereby when said emission surface is coupled with the treatment region a therapeutic affect is generated with the treatment region.

9. A phototherapy system as recited in claim 8, wherein said therapeutic affect is at least one from the group including photodynamic therapy, photodynamic acne therapy, photodynamic psoriasis treatment, bacterial sterilization, wound healing, treatment of cutaneous T cell lymphoma, treatment of vitiligo, treatment of fungal infection, treatment of lichen planus, treatment of granuloma annulare, treatment of warts, treatment of pityriasis rosea, treatment of generalized itching from various causes, treatment of atopic and other types of eczema, treatment of non-melanoma skin cancer, photorejuvination, treatment of hidradenitis suppurativa, treatment of Lichen sclerosus, treatment of scleroderma, treatment of alopecia areata, treatment of lichen planus, treatment of Darier's disease, treatment of vulvar intraepithelial neoplasia, treatment of cervical intraepithelial neoplasia, treatment of penile intraepithelial neoplasia, treatment of anal carcinoma in situ, treatment of Barrett's esophagus, treatment of oral candidiasis, anti viral therapy, hair removal, treatment of Bowen's disease, treatment of oral cancer, treatment of actinic keratosis, treatment of Leukoplakia, treatment of sun damage, cosmetic skin improvement, treatment of oily skin, treatment of enlarged sebaceous glands, and treatment of wrinkles, treatment of cancer.

10. A phototherapy system according to claim 1, whereby when said emission surface is coupled with the treatment region said optical device blocks said emitted light from areas outside the treatment region.

11. A phototherapy system according to claim 1, whereby when said emission surface is coupled with the treatment region said optical device blocks exogenous light from areas within the treatment region.

12. A phototherapy system as recited in claim 1, further comprising said photoactive compound within the treatment region.

13. A phototherapy system as recited in claim 12, wherein said photoactive compound is at least one from the group including photosensitizers, fluorophores, chromophores, semiconductors, nanoparticles, and plasmon nanoparticles.

14. A phototherapy system as recited in claim 1, further comprising a treatment patch having a top patch surface and a bottom patch surface.

15. A phototherapy system as recited in claim 14, wherein said treatment patch includes a translucent region through which to align said patch to the treatment region.

16. A phototherapy system as recited in claim 14, wherein said treatment patch includes an adhesive on said bottom patch surface to attach said patch to the treatment region.

17. A phototherapy system as recited in claim 14, wherein said treatment patch includes an adhesive on said top patch surface to attach said optical device to said treatment patch.

18. A phototherapy system as recited in claim 14, wherein said treatment patch includes a treatment aperture.

19. A phototherapy system as recited in claim 18, wherein said treatment patch includes a light blocking region around said treatment aperture.

20. A phototherapy system as recited in claim 14, wherein said treatment patch includes a treatment compound.

21. A phototherapy system as recited in claim 20, wherein said treatment compound includes at least one from the,group including anesthetic, photoactive compound, index matching material and thermal compound.

22. A phototherapy system according to claim 1, wherein said emission surface has a radiant emittance greater than 62.8 milliWatts per square centimeter.

23. A phototherapy system according to claim 1, wherein said wavelength is within the range of at least one from the group including 400-410 nm, 630-635 nm; 660-700 nm, and 790-810 nm.

24. A phototherapy system according to claim 1, wherein said light source is a remote light source tethered by an optical waveguide.

25. A phototherapy system according to claim 1, wherein said emission surface is a conformable surface that conforms to the treatment surface.

26. A phototherapy system for a treatment region having a treatment surface, comprising: an optical device including a light source generating source light and an emission surface emitting emitted light, said emission surface having an initial radiant emittance; and whereby when said emission surface is coupled with the treatment region, said emitted light in combination with reflected light and scattered light from the treatment region sum to create an irradiance on the treatment surface that is greater than 1.5 times said initial radiant emittance.

27. A phototherapy system as recited in claim 26, wherein said optical device further includes light extraction features, wherein said light extraction features have an extraction feature distribution producing said total light intensity distribution that is homogenous over the treatment region.

28. A phototherapy system for a treatment region having a treatment surface, comprising:
a) an illuminating layer having a light source and an emission surface emitting emitted light;
b) a treatment patch having a top patch surface, a bottom patch surface and a treatment aperture therein, said illuminating layer couple to said top patch surface such that said emitted light from said emission surface enters said aperture; and
c) wherein said emitted light has a wavelength to activate a photoactive compound, said emitted light having a therapeutic radiant emittance.

29. A phototherapy system as recited in claim 28, wherein said treatment patch includes a translucent region through which to align said patch to the treatment region.

30. A phototherapy system as recited in claim 28, wherein said treatment patch includes adhesive on said bottom patch surface to attach said patch to the treatment region.

31. A phototherapy system as recited in claim 28, wherein said treatment patch includes adhesive on said top patch surface to attach said illuminating layer to said treatment patch.

32. A phototherapy system as recited in claim 28, wherein said patch includes a treatment compound.

33. A phototherapy system as recited in claim 28, wherein said treatment patch includes a light blocking region around said aperture.

34. A method of delivering phototherapy to a treatment region having a treatment surface, comprising the steps of:
a) providing i) a light source generating source light with a source radiant emittance, ii) a detector, and iii) a feedback loop between said detector and said source light;
b) coupling said light source with the treatment region so that said source light in combination with reflected light and scattered light from the treatment region sum to create a combined radiant emittance greater than 1.5 times said source radiant emittance;
c) generating a desired fluence rate from said source light upon the treatment surface;
d) measuring fluence rate provided to the treatment region by said detector; and
e) delivering a desired light delivery protocol to the treatment surface based on feedback from said feedback loop.

35. A phototherapy system as recited in claim 34, further providing light extraction features, wherein said light extraction features have an extraction feature distribution producing said total light intensity distribution that is homogenous over the treatment region.
